(19) United States
(12) Patent Application Publication
(10) Pub. No.: US 2011/0301472 A1
(43) Pub. Date: Dec. 8, 2011

(54) METHOD AND APPARATUS FOR DETERMINING AND/OR MONITORING A PHYSICAL CONDITION OF A PATIENT BASED ON AN AMPLITUDE OF A PRESSURE SIGNAL

(75) Inventors: Tobias Grober, Heusenstamm (DE); Ulrich Moissl, Bad Vilbel (DE); Peter Wabel, Darmstadt (DE)

(73) Assignee: FRESENIUS MEDICAL CARE DEUTSCHLAND GMBH, Bad Homburg (DE)

(21) Appl. No.: 13/133,048

(22) PCT Filed: Dec. 8, 2009
(86) PCT No.: PCT/EP2009/008765

§ 371 Date: Aug. 17, 2011

(30) Foreign Application Priority Data
Dec. 9, 2008 (DE) 10 2008 061 122.0

(51) Int. Cl. A61B 5/021; A61B 5/08; A61B 5/024; A61B 5/00

(52) U.S. Cl. 600/485; 600/300; 600/529; 600/500

(57) ABSTRACT

A method for determining and/or monitoring quantities, in particular cardiovascular quantities, relating to a patient's condition, and an apparatus for measuring an amplitude of a cardiac pressure signal are disclosed. The amplitude of the pressure signal may be detected with the aid of a pressure sensor of a blood treatment apparatus, and its magnitude may be corrected by the contribution of the blood pump of the blood treatment apparatus so as to determine the amplitude of the cardiac pressure signal of the patient. The value of the amplitude of the pressure signal thus determined may subsequently be evaluated.

### FIELD OF THE INVENTION

The present invention concerns a method for determining and/or monitoring a physical condition of a patient and an apparatus for carrying out the method of the present invention. It furthermore concerns an apparatus for determining and/or monitoring a physical condition, in particular at least one cardiovascular quantity, of a patient and a treatment apparatus including the apparatus for determining and/or monitoring the physical condition.

### BACKGROUND

In practice, suitable quantities are determined and evaluated for the purpose of determining and monitoring a patient's physical condition. On the basis of these it is possible to draw conclusions as to the patient's condition at a past time, at a time of measurement, or at a later time; in a given case, action is taken accordingly. The like methods and apparatus employed for this purpose are known for the heart rate—as one example of a determined and evaluated quantity—from European Patent Application Publication EP 0 330 761 A1 and from European Patent Publication EP 0 957 956.

It is the object of the present invention to specify another method as well as suitable apparatus for determining and/or monitoring a physical condition of a patient.

### SUMMARY

The object of the present invention is achieved through a method having the features described herein. The method of the present invention includes an evaluation of an amplitude of a pressure signal. In the framework of the present invention, an amplitude of a pressure signal should be understood not as a signal but as a single value, and in particular the magnitude thereof.

As was stated in the foregoing, detecting the heart rate for the purpose of assessing a patient's physical condition is already known. It was now surprisingly found in studies carried out by the applicant on amplitudes of pressure signals of 50 patients undergoing a blood treatment, that an “amplitude of a pressure signal” may advantageously also be used for assessing the patient's physical condition. In this context, according to the present invention an “amplitude of a pressure signal” is to be understood as an amplitude of a pressure signal measured on a patient's body or extracorporeally—e.g., in the course of a medical treatment—or determined from such measurements, in particular an amplitude of a cardiac pressure signal of the patient or an approximation thereof. Just like the heart rate, the amplitude of a pressure signal and in particular the amplitude of a cardiac pressure signal or the evolution thereof, respectively, may be subject to fluctuations or variations, for instance during a dialysis treatment of the patient or also across extended periods of time.

In a dialysis treatment the causes for this may be variations of the heart's stroke volume, of the fistula pressure, of the degree of filling of the fistula or of the vascular system, respectively, of the placement of the needle, an arterial underpressure in the tubing system of the blood treatment apparatus, a developing in- or outflow stenosis of the fistula—in particular with Goretex grafts—vascular stiffness, elasticity, calcification of the fistula and/or of the supplying/discharging blood vessels, or reflections of the pulse wave in the vascular system and others.

In dialysis patients just like in non-dialysis patients, additional reasons may lead to a fluctuation or variation, or favor these. The fluctuations of the amplitude of the pressure signal and particularly of the amplitude of the cardiac pressure signal may be occasioned by the stroke volume of the heart, the heart time volume, the discharge volume, the contractility, heart valve defects, the vessel status—particularly in diabetes patients—or variations of these. The fluctuations or variations may furthermore be occasioned by technical circumstances or other circumstances such as location of punction, thickness of the needle, compliance of a utilized system, a change in the patient's position, movements of the patient, recirculation, hydrostatic pressure increases, variations of the TPR (Total Peripheral Resistance or vascular resistance in peripheral blood vessels), or the like. The evaluation of the amplitude of the pressure signal and above all of the amplitude of the cardiac pressure signal may advantageously be employed for assessing the above-mentioned as well as further phenomena or variations in the sense of a “physical condition”—to be understood in the present meaning. In dialysis patients in particular, the estimation of the stroke volume or heart time volume (cardiac output) of the heart by means of an evaluation of the amplitude of the pressure signal may be of high interest.

In accordance with the present invention, an “amplitude of the pressure signal” may designate the determined or measured value of the pressure signal—or its magnitude—in a maximum excursion or in a maximum, respectively. The maximum excursion may be defined as the difference between the value of the maximum pressure peak and a base line, usually a mean pressure such as, e.g., 0 mmHg. The amplitude of the pressure signal may moreover be established in some other manner.

“Determination and/or monitoring of at least one physical condition” or of a quantity relating to a patient's physical condition (where “physical condition” and “quantity relating to the physical condition” may also be used synonymously in the following) may be performed for assessing both a pathological condition and a non-pathological, in particular physiological, condition of the patient. It may be performed for monitoring the latter and/or for accompanying or monitoring a treatment of the patient and/or for diagnostic purposes.

Determination and/or monitoring may take place without a comparison of the amplitude of the pressure signal or of the evaluation results with reference values of other patients. Thus, the observation of an intra-individual development or variation of the amplitude of the pressure signal of one and the same patient across a period of time may already result in a statement concerning the physical condition thereof. A comparison with reference data of third persons is evidently not necessary for this purpose, however is possible and also provided in a preferred embodiment.

“Determination and/or monitoring” may take place during a treatment of the patient, however also at a later time in the patient's absence, or without the patient still being connected to a means for determining the amplitude of the pressure signal and/or to a treatment apparatus. Moreover it is not necessary for the amplitude of the pressure signal to have been determined on the patient in the narrower meaning of the expression. The amplitude of the pressure signal may also have been determined extracorporeally on a blood pump, a blood circulation, or the like.

A “quantity relating to the physical condition of a patient” or a “physical condition” may be any quantity, in particular physiological quantity, that is suited as a characteristic quantity for evaluating and characterizing a patient's physical condition or a partial aspect thereof. Examples of this encompass cardiovascular quantities such as fistula blood flow in a dialysis patient with an applied fistula or shunt, a heart rate, a cardiac pressure amplitude, a fistula condition, an arrhythmia, the operation of a heart pacemaker and respiratory quantities, however without being restricted to these. A respiratory quantity may, e.g., be a respiration signal such as the respiration frequency, a pathological respiration pattern such as a paradoxical respiration, and the like. In terms of the present invention, however, the physical condition should not a priori be understood to be the amplitude of the pressure signal and in particular not the amplitude of the cardiac pressure signal. Only its evaluation, not already its measurement or determination, allows an inference of the physical condition within the meaning of the present invention.

Although various passages of the present description exemplarily relate to dialysis patients, the present invention is certainly not restricted to such dialysis patients.

A “patient” within the meaning of the present invention may be a member of any species (human or animal), irrespective of whether in good or bad health.

“Evaluation of the amplitude of the pressure signal” may be performed, e.g., with the aid of corresponding—and optionally different—evaluation means and/or evaluation methods. “Evaluation of the amplitude of the pressure signal” may thus involve an accurate evaluation of the magnitude or evolution of the amplitude of the pressure signal—for instance by means of various calculating operations—as well as an estimation of the magnitude or evolution of the amplitude of the pressure signal, or of a trend thereof based on the determined amplitude of the pressure signal or magnitude or evolution, respectively. The evaluation that will be required in the individual case may differ from patient to patient; a person having skill in the art may determine in the light of the given circumstances what kind of evaluation appears to be suitable for optimal care of the patient. Examples of an evaluation will be given further below. An association with particular clinical conditions or symptoms is not required in order to carry out the method of the present invention and is furthermore not provided. The method of the present invention rather serves the purpose of obtaining characteristic figures, parameter values, etc. This particularly also applies to the evaluation step.

For the “evaluation” of the amplitude of the pressure signal it is possible in accordance with the present invention to use any information that is discernible in or may be deduced from the amplitude, as well as any information that may be may be deduced from the measurement or determination conditions. Thus the very magnitude of the amplitude may enable a statement. Even a magnitude of the amplitude compared to amplitudes of other patients may enable a statement; its comparison with the like data and the extraction of insights gained from this may accordingly also constitute an evaluation within the meaning of the present invention. In addition, the evolution of the amplitude may furnish information on the physical condition. The respective evolution may correspond to a representation of several heart amplitude values over time or may be extracted from the latter. A time difference (=1/sampling rate, thus corresponding to the reciprocal value of the sampling rate) between individual heart amplitude values may amount, e.g., to one half of the time window (here: 5 seconds) across which the amplitude was determined. “Evaluation” may also be understood to be the mean amplitude of the pressure signal and in particular of the cardiac pressure signal, for example during a treatment session of the patient or during some other time period. If, for example, quasi-stationary values for the amplitudes of the pressure signal were determined across 10-second sections during a treatment, it is then possible to form the average or median value through these values (i.e. across their evolution), whereby one thus obtains for each treatment—or for one time period in general—a value that may be observed across an extended period of time (weeks/months). An evaluation of this value can also enable statements concerning the physical condition. Furthermore it may be possible to establish trends. “Evaluation” does, of course, also cover a comparison of an evolution of the amplitude with evolutions in other patients.

In accordance with the present invention, it may be preferred to evaluate the amplitude of the pressure signal by itself. For determining and/or monitoring it is, however, also possible to consider an additional quantity. In this case the quantities may be measured at identical or different points of time. The quantities may be directly interrelated or be independent of each other.

A like quantity—just like the amplitude of the pressure signal—may furthermore also be determined and/or monitored not by immediate detection as a measurement value but by evaluation, filtering, conversion etc. of another quantity, in particular a measured one.

This other quantity may be a quantity that is accessible by a different path. The evaluation as mentioned above may, for example, be supplemented by measuring and evaluating additional quantities such as heart rate, mean fistula pressure ((Pₐᵣₜ(arterial fistula pressure)+Pᵥₑₙ (venous fistula pressure))/2) or other parameters, in particular dialysis-specific parameters.

In order to enable meaningful conclusions, an “evaluation” need not necessarily furnish exact values. In a given case it may also be sufficient to determine an approximation to the actually existing, exact values. By this, too, it may be possible to observe an evolution of the determined values and thus a trend thereof. Such an approach may serve to further reduce the complexity in the evaluation of the determined quantity. Both the expenditure in terms of apparatus and a calculation effort required for the evaluation of the amplitude of the pressure signal may thus advantageously be reduced.

In the description of the present invention, the expression “pressure signal” may be understood to be a continuous pressure signal if sampled at a sufficiently high rate (e.g., 20 Hz). This signal may be subdivided into smaller time periods of, say, 10 seconds, in which frequency and amplitude are considered to be constant (in this case referred to as a stationary signal). The pressure signal may be composed of different signals. One component of the pressure signal may be a cardiac pressure signal, i.e., a signal engendered by the contraction of the heart. One more component may be a pump pressure signal, i.e., a pressure signal engendered by a blood pump, in a given case a continuous one.

In accordance with the present invention, the “amplitude of the pressure signal”—or also of a cardiac pressure signal—is understood to be the amplitude of a stationary or approximately stationary (quasi-stationary) (cardiac) pressure signal section, for example across a time window of 10 seconds. With the aid of a histogram such as shown in FIG. 8, one accordingly obtains an estimation of a single heart amplitude value across the time window that was used for filling the histogram (the time window may have a duration of between 1 and 5 minutes, for instance).

Advantageous developments of the present invention are further described herein.

The amplitude of the pressure signal evaluated by means of the method of the present invention may be determined during a blood treatment session using a blood treatment apparatus. It may in particular be determined or measured extracorporeally.

A “blood treatment apparatus” may be employed for a blood treatment and/or blood purification and be configured as a dialysis apparatus or an infusion pump connected to the patient's vascular system, e.g., by means of shunt, fistula or catheter. A dialysis apparatus may, inter alia, be a means for a hemodialysis, hemofiltration, or hemodiafiltration. Such means as a general rule include an extracorporeal blood pump.

In order to enable meaningful conclusions, a “determination” need not necessarily supply accurate values. As a rule it is sufficient to merely know a level or a trend thereof. Even an approximation to the actually existing values may be sufficient in a given case. Such a simplified approach may further reduce the complexity involved in determining the relevant quantity. The expenditure in terms of apparatus for determining the quantity, in particular a measurement or calculation effort, may thus advantageously be reduced.

An “amplitude of a pressure signal” may be measured in the extracorporeal blood circulation by using one or several of the pressure sensors that are provided in a blood treatment apparatus.

Suitable pressure sensors are generally known in the prior art and include—without being restricted to these—piezoelectric, piezoresistive, frequency-analogous, capacitive, inductive pressure sensors and/or pressure sensors with Hall elements, as well as combinations of these.

Such a pressure sensor may be integrated in the arterial or venous branch of the tubing system or other sections, in particular on the blood pump of the blood treatment apparatus.

Besides the desired amplitude of the pressure signal which is, for example, the actual amplitude of the patient's cardiac pressure, the amplitude of the pressure signal may encompass further, undesirable pressure signals such as, e.g., parasitic signals which may originate in additional means employed in the blood treatment apparatus, and/or measurement noise.

These additional amplitudes of pressure signals that may in a given case be undesirable in evaluation may be eliminated from the relevant signal component by means of known methods.

Measurement noise, for instance, may be eliminated with the aid of a simple bandpass filter.

In a preferred embodiment, the method therefore includes a step of correcting the value of the amplitude of the pressure signal by a contribution of the blood treatment apparatus to the magnitude of the amplitude of the pressure signal.

Such a contribution may, for example, be an amplitude of the pressure signal originating in an extracorporeal blood pump used in the blood treatment, or the magnitude thereof.

“Correcting the amplitude of the pressure signal” may be enacted in accordance with the method known from European Patent Application Publication EP 0 330 761 A1 and from European Patent Publication EP 0 957 956 B 1, the related contents of disclosure of which are hereby fully incorporated by way of reference.

In addition, it is also possible to use the method known from the doctoral thesis by one of the inventors of the present invention, Mr. Ulrich Moissl, entitled “Kardiovaskuläre Überwachung bel der Hämodialysetherapie” [Cardiovascular Monitoring in Hemodialysis Therapy], Technische Universita Darmstadt, Germany, 2005. The related contents of disclosure of the doctoral thesis are hereby also fully incorporated by way of reference. According to the approaches described there, the amplitude of the cardiac pressure signal is determined during the patient's extracorporeal blood treatment at exemplary intervals of 10 seconds each while taking a current signal model of the blood pump into consideration. This allows simulation of the temporal evolution and of the amplitude of the pressure signal in a manner close to reality. An on-line method is equally described in the doctoral thesis, which outputs the new cardiac pressure signal value at each new sampling point (thus, e.g., 20 times per second). The algorithms used for this purpose are described in the above-identified doctoral thesis. The related contents of the doctoral thesis are therefore presently also fully incorporated by way of reference.

From German Patent Publication DE 101 15 991 C1, European Patent Application Publication EP 0 330 761 A1, European Patent Publication EP 0 957 956 B1, and U.S. Patent Application Publication No. 2005/0010118 A1, methods for the separation of heart signal and blood pump signal are known wherein the signals are separated based on different frequency spectra. The related contents of this (patent) literature are presently also fully incorporated by way of reference.

Correction of the magnitude of the amplitude of the pressure signal may be carried out simultaneously with its measurement or at a later point of time.

It may, for example, take place automatically at particular predetermined times.

The intervals between the times may vary depending on external conditions or measurement results.

A further preferred embodiment of the method provides to correct the value of the amplitude of the pressure signal as a function of a signal transmitted by at least one position sensor. The position sensor enables sampling of the cardiac pressure signal at a constant rotary angle of the pump rotor.

The position sensor may be a Hall sensor—or may co-operate with the latter—which outputs a pulse, but may also be configured as an optical sensor adapted, e.g., to recognize a black line on the rotor, or in any other manner that is known to the person having skill in the art. A “Hall sensor” is a means for measuring a voltage in an energized conductor that is located inside a stationary magnetic field. The operation of Hall sensors is described, e.g., in the paper by Josef Janisch, “Was Sie schon immer fiber Hallsensoren wissen wollten: Kleiner Effekt—Groβe Wirkung” [What you always wanted to know about Hall sensors: Small effect—Momentous result], pp. 1 to 5, elektronik industrie 7, 2006.

According to the present invention, the Hall sensor(s) may be employed according to the description in German Patent Application Publication DE 102 30 413 A1, the related contents of disclosure of which are hereby fully incorporated by way of reference.

“Correction of the value of the amplitude of the pressure signal as a function of a transmitted signal or output pulse from a position sensor, in particular a Hall sensor” has the meaning, for example, that the amplitude of the determined pressure signal is corrected by a contribution of the blood treatment apparatus, transmitted at a particular time by a sensor as a result of a pulse generated by the Hall sensor, to the magnitude of the amplitude of the pressure signal.

Such a contribution may be a magnitude of an amplitude of the pressure signal originating in the blood pump. Other considerations of the Hall sensor signal are, of course, also possible in the framework of the present invention. This is particularly true for any methods already known to the person having skill in the art.

In this embodiment, sampling is performed at each pump rotation in a respective identical rotor position. In this way the pressure signal is effectively undersampled with regard to the relevant heart signal components. A continuous calculation of the amplitude of the cardiac pressure is here not possible owing to the undersampling; however, one obtains a sufficient number of values of the evolution of the pressure signal, and in particular of the cardiac pressure signal, that is considered to be constant, e.g., across several minutes.

The amplitude representing the maximum value of the pressure signal may be estimated from the determined pressure signal values. If determination is always carried out at a maximum value of the pressure signal, one thus implicitly even obtains its amplitude.

Particularly when represented accordingly, for example by means of histogram, they allow a good estimation of the amplitude. As such values may be acquired several times during a blood treatment session—and furthermore across several such sessions—for example their standard deviation—and optionally their averaging or otherwise suitable mathematical processing—constitutes a good estimate, e.g., for the mean amplitude across the duration of treatment. This particularly allows a good identification of trends, i.e., long-term developments.

When the pump operates with sufficient uniformity and the rotation period is known with sufficient accuracy, the histogram may also be constructed by time-synchronous sampling.

In another preferred embodiment, the method includes a correction of the magnitude of the amplitude of the pressure signal as a function of a rotary angle of a blood pump of the blood treatment apparatus, in particular a peristaltic blood pump. A “peristaltic blood pump” is a positive-displacement pump customarily employed in a blood treatment and/or blood purification method for transporting the bloodstream in the extracorporeal blood circulation. It may, for instance, have the form of a scroll pump.

“Correcting the value of the amplitude of the pressure signal as a function of a rotary angle of a peristaltic blood pump” means that the amplitude of the pressure signal is corrected by a particular signal of the blood pump that correlates with a particular rotary angle of the blood pump. In other words, the respective amplitudes are measured at a particular rotary angle of the blood pump and/or corrected by taking the rotary angle into account. This is an advantageous option particularly with blood pumps of insufficiently uniform rotation. Particularly in the case of angle synchronous sampling this is advantageously possible.

In a preferred development, the method includes an evaluation of the value of the corrected amplitude of the pressure signal by comparing the amplitude to predetermined reference values.

The “predetermined reference values” may originate from the same patient and/or may be values obtained from other patients and/or experience values and/or values of persons that are not conspicuous in pathological respect.

Such an evaluation may take place by comparing exact values. It may, however, also be sufficient to observe a particular trend of the corrected amplitude of the pressure signal or of its magnitude with regard to the known reference values.

On the basis of a corresponding evaluation it may, for example, be possible to detect a current physical condition of the patient at the time of evaluation and, based on the obtained results, draw conclusions to possible critical situations or situations requiring action, and in a given case to initiate measures in due time.

Repeated evaluation of the amplitude may furthermore serve for imaging a course of treatment or to furnish evidence for a successful treatment.

A further preferred development of the method of the present invention includes the extracorporeal determination or measurement of the amplitude of the pressure signal. In a further preferred manner, the method of the present invention is performed as an off-line method, so that the patient's continued presence advantageously is not required for the determination or the evaluation. For instance, the patient also need not remain connected to a treatment apparatus for the evaluation.

However an on-line performance of the method of the present invention as well as combinations of on-line (e.g., for data acquisition) and off-line (e.g., for data evaluation) are encompassed by the present invention.

In a further preferred embodiment of the method of the present invention, the monitored or determined quantity is a respiration signal.

In patients with a heart catheter, the respiration signal may be determined by measurements in the patient's right atrium and may relate, for instance, to the respiration frequency and/or to the respirational depth of one or several breaths.

By means of determining and/or monitoring the respiration signal based on the knowledge of the amplitude of the pressure signal and in particular of the amplitude of the cardiac pressure signal it is possible, for example, to image a respiration profile of the patient and thus detect respiration fluctuations or irregular respiration such as, e.g., Cheyne-Stokes respiration.

In the same way it is possible to detect sleep apnea by means of the method of the present invention.

In a preferred development, the method of the present invention furthermore includes an evaluation of the amplitude of the pressure signal for the observation of a long-term trend of quantities, in particular cardiovascular quantities.

“Observing the long-term trend” may encompass a time period of several, few hours up to some weeks and/or months. In the case of a dialysis patient, such a time period may encompass a plurality or multiplicity of dialysis treatments.

It may thus be possible in accordance with the present invention to image a temporal evolution of a patient's physical condition. Thus, for example, a fistula and in particular a new placement of a fistula may be monitored across several months with a concurrent evaluation of the pressure amplitude signal in order to recognize variations at the vascular access prior to these turning critical or requiring treatment, respectively. In this way it may advantageously be possible to do away with complex and partly costly interventions or new placements of fistulae, respectively. Developing stenoses may optionally be recognized and dilated in a timely manner, for instance by using balloon catheters.

To this end it may be advantageous to detect the amplitude of the patient's cardiac pressure signal—optionally in addition to a variation of the heart rate, of the mean fistula pressure or a variation thereof, and/or other dialysis-specific variables or their variation—in long-term monitoring.

In a preferred development, the method of the present invention may be employed during a blood treatment, in particular a hemodialysis, a hemofiltration, or a hemodiafiltration.

Here it may be advantageous to utilize the measurement and/or evaluation means of the blood treatment apparatus, which already were provided for the blood treatment, in order to carry out the method of the present invention.

It is furthermore preferred to employ the method of the present invention for an evaluation of the determined amplitude of the pressure signal as to tachycardia, bradycardia and/or hyper- or hypotensive episodes.

In a development of the present invention, the method of the present invention serves for establishing a classification of a patient with the associated known advantages. In particular it is possible to enhance the accuracy of the statement concerning an individual patient by classification.

In further preferred embodiments of the present invention, the quantity to be determined and/or monitored is a heart rate and/or an arrhythmia and/or the operation of a heart pacemaker. By determining and/or monitoring an arrhythmia it is, for instance, possible to recognize extrasystoles or syncopes.

It is thus possible, e.g., to suitably adapt a dialysate concentration, for instance by increasing its potassium content or otherwise modifying its composition.

In a further preferred embodiment, a Fourier spectrum of the heart amplitudes (not to be confused with the cardiac pressure signal) may be formed over the course of a treatment or a treatment section (e.g., 10 to 30 minutes). Hereby it is possible to detect rhythmical variations of the heart signal amplitude. Under the viewpoint of a continuous 20-Hz cardiac pressure signal, this would amount to an amplitude modulation. Here it might be possible, for instance, to speak of a “heart amplitude variability” in analogy with the “heart rate variability.”

This manner of proceeding might, e.g., reflect the influence of the hormonal blood pressure regulation of the baroreceptor control loop or of similar long-term control loop oscillations, but also a short-term beat-to-beat modulation of the blood offered in the fistula in a case where heart and pump (scroll pumps draw in a pulsatile manner, not continuously) are not “beating” or transporting in synchronicity.

It is therefore conceivable that initially the pump draws blood, with the next wave of blood only entering the fistula milliseconds later.

The present invention is not restricted to the quantities that were presently indicated by way of example. Where this is of interest, it is also possible to determine and/or monitor further quantities, in particular physiological ones, that were presently not mentioned.

The object of the present invention is furthermore achieved through an apparatus as described herein. The apparatus of the present invention may include the respective means required and suited for performing the method of the present invention in each one of its embodiments. In order to avoid repetitions of the functions of the single components, elements, and/or advantages, reference is made to the components, elements and/or method steps explained in connection with the method of the present invention. The advantages achievable by way of the method of the present invention may be achieved undiminished by means of the apparatus of the present invention.

The object of the present invention is furthermore achieved through an apparatus as described herein. This apparatus of the present invention comprises at least analogous means for evaluation and optionally measurement of an amplitude of a pressure signal.

In each apparatus of the present invention, a means for determining and/or monitoring the quantities and/or a means for evaluating the amplitude of the pressure signal may be a means that is known from the prior art and suited for this purpose.

The means of each apparatus of the present invention may be, or include, automated means and/or means for data processing such as, e.g., a CPU.

The amplitude of the pressure signal may be evaluated statistically with the aid of corresponding means. Just like the heart frequency, it may be extracted with the aid of corresponding means during a dialysis treatment from the amplitude of the pressure signal measured on the extracorporeal blood circulation and in a given case recorded.

In a preferred embodiment, the apparatus of the present invention furthermore includes at least one means for correcting the value of the amplitude of the pressure signal by a contribution of a blood treatment apparatus to the magnitude of the amplitude of the pressure signal as a function of a signal or pulse of at least one position sensor or Hall sensor and/or as a function of the rotary angle or some other suitable technical quantity of a blood pump of the blood treatment apparatus.

Such a contribution may be an amplitude of the pressure signal of a blood pump. Correction of the amplitude of the pressure signal is preferably performed in an automated manner.

The time for detecting the contribution of the blood treatment apparatus to the magnitude of the amplitude of the pressure signal may correspondingly be predetermined by a particular signal of at least one Hall sensor and/or a particular rotary angle of a blood pump of the blood treatment apparatus.

In the case of Hall sensor-synchronous sampling, the pump signal may already be corrected implicitly. In the optimal case, the pressure contribution of the pump is always identical and amounts to −180 mmHg, for example. This value also includes the mean fistula pressure. As the latter is not known with precision, the histogram may be standardized to 0.

Where the contribution of the blood pump is known, however, it is possible to calculate the fistula pressure on its basis: With identical or comparable geometry of pump, needle and tube, etc. and with given blood viscosity/given hematocrit it is possible to plot a pure pump pressure curve in the laboratory.

The difference between its evolution and an evolution of the measured values or of the pressure signal, respectively, is the fistula pressure, if only all of the other conditions correspond to the lab environment; otherwise it is possible with the above-identified approach to obtain an approximation of the fistula pressure or of its evolution.

In a development of the present invention, the apparatus furthermore comprises a means for supplying reference values.

This means may serve for storing the reference values and may be a storage means as is usual in the prior art and suited for this purpose, such as, e.g., a ROM, a RAM, a disc, a memory card, a USB stick, etc.

The apparatus of the present invention may furthermore comprise further means for filtering parasitic signals and/or for analyzing and/or converting the amplitude of the determined pressure signal in order to obtain the desired signal of the patient.

This filtering may take place in accordance with the description given in the above-mentioned doctoral thesis. The respective contents thereof are herewith incorporated by reference.

The object of the present invention is furthermore achieved through a blood treatment apparatus as described herein. Such a blood treatment apparatus includes at least one apparatus of the present invention as described in the foregoing. A blood treatment apparatus may, for example, have the form of a dialysis apparatus as presently described at the outset in connection with a blood treatment.

The method of the present invention and the apparatus of the present invention advantageously allow to determine or monitor at last one quantity relating to the physical condition of a patient, in particular a cardiovascular quantity. According to the present invention, this may be done without particular complexity and moreover non-invasively. Its realization is furthermore possible without considerable additional expenditure in terms of apparatus, in particular during a blood treatment. The method of the present invention advantageously allows recognition of in particular a variation at a vascular access (e.g., fistula or shunt). This is particularly true in long-term monitoring of the amplitude of the pressure signal. For the performance of the method of the present invention and/or for the utilization of the apparatus of the present invention, special schooling and/or training of the hospital personnel and/or of clinical personnel is advantageously not necessary. The method of the present invention is thus characterized by its simple/easy execution and comparatively simple evaluation. This is particularly true for a measurement of the amplitude of the pressure signal at a particular predetermined point of time of a Hall sensor signal and/or rotary angle synchronously with the blood pump provided in the blood treatment apparatus, and correction of the determined amplitude of the pressure signal by a known contribution of the blood treatment apparatus to the signal, e.g., by the contribution of the blood pump. When the amplitude of the pressure signal of the blood pump at a particular rotary angle is known, the correction may advantageously be simplified further, for a respective, substantially identical contribution may be assumed for the value of the contribution of the blood pump to the magnitude of the amplitude, and the respective determined amplitude of the pressure signal may be corrected by this known value in order to obtain the desired amplitude of the patient's heart signal or an approximative value. This may be of advantage particularly when the rotation of the blood pump is not sufficiently uniform. The possibility of recognizing calcifications inside a fistula or inside the patient's vascular system is a further advantageous possible application of the present invention.

Even where the amplitude of the pressure signal of the blood pump is not known, it is possible to determine the amplitude of the cardiac pressure signal if the contribution of the pump is always the same (which is ensured by the Hall sensor-synchronous sampling at an invariably same rotor position) or at least approximatively the same.

If undersampling takes place at a time at which the pump signal does not change substantively, i.e., if the derivation is close to 0 (e.g., immediately preceding engagement of one of the two pump scrolls in the tubing system in the case of a scroll pump), the measurement necessitates no technical complexity or only a low one. Such manner of proceeding may be enabled by a favorable placement of the Hall sensor magnet on the rotor. The influence of the pump signal advantageously is minimized further in this case.

The evaluation of the amplitude of the pressure signal, of the amplitude of a patient's cardiac pressure signal or of an approximative value thereof may advantageously be used for locating stenoses and/or assessing the condition of a fistula and for the timely avoidance of critical conditions. By comparing the desired characteristic quantities to reference values and/or observing a trend of the quantities it is possible to prevent the development of a pathological condition and/or preclude a deterioration towards a critical condition. Performance of the method of the present invention does not cause any discomfort to a patient, in particular if the method—which may also be used on-line—is used off-line, which represents one advantage of off-line performance. Another advantage of the method of the present invention resides in the fact that the patient does not have to be present at the time of evaluation. The combination of the method of the present invention with a blood treatment such as, e.g., a hemodialysis, hemofiltration, or hemodiafiltration, as well as the combination of the apparatus of the present invention with a blood treatment apparatus suited for this purpose requires hardly any expenditure in terms of apparatus and may thus save both time and costs while in addition advantageously also avoiding further discomfort to the patient.

Just the same it is advantageously not only possible to evaluate the directly measured quantities for the assessment of a condition, but based on the directly measured quantities it is also possible to draw conclusions concerning other quantities of interest. Thus, for instance, there exists an interrelation between fistula pressure and pulse amplitude. If the pulse amplitude becomes lower during a treatment, for example, this may be due to a reduction of the stroke volume on the one hand and a reduction of the fistula pressure on the other hand. Accordingly it may be possible to consider the effect of the stroke volume via the detected heart rate by calculation to thus infer a relative variation of the fistula pressure. Furthermore it may be possible to infer the blood offered in the fistula via a determination of the amplitude of the pressure signal at various speeds of the blood pump to thus estimate the fistula flow in a given case.

Although in the foregoing the method of the present invention was described in connection with a blood treatment apparatus, the present invention is not restricted to a use with a patient being subjected to a blood treatment and/or blood purification by means of a corresponding apparatus.

The present invention shall be further described exemplarily by making reference to the annexed drawings.

### BRIEF DESCRIPTION OF THE DRAWINGS

FIG. 1 schematically shows the principle of heart signal extraction.

FIG. 2 shows graphs of a heart rate (top) and of an amplitude of a cardiac pressure signal (bottom) versus time.

FIG. 3 shows value ranges of amplitudes of the cardiac pressure signal of a patient versus time, indicated for the months of February (02/08) to September (09/08) of the year 2008.

FIG. 4 shows graphs of a heart frequency (top) and of an amplitude of a cardiac pressure signal (bottom) versus time.

FIG. 5 shows a graph representing a superposition of the influences of respiration and of the amplitude of the cardiac pressure signal versus time.

FIG. 6 shows a graph indicating a Cheyne-Stokes respiration.

FIG. 7 shows further graphs of a heart frequency (top) and of an amplitude of a cardiac pressure signal (bottom) versus time.

FIG. 8 schematically shows the extraction of a cardiac pressure signal (top) and a corresponding representation in the histogram (bottom).

FIGS. 9a to 9c schematically show the deviation of a pump frequency from a target frequency (FIG. 9a) and the difference between time-synchronous (FIG. 9b) and angle-synchronous (FIG. 9c) sampling of the pump signal.

FIG. 10 shows a graph of an amplitude of a cardiac pressure signal versus a corrected fistula pressure.

### DETAILED DESCRIPTION

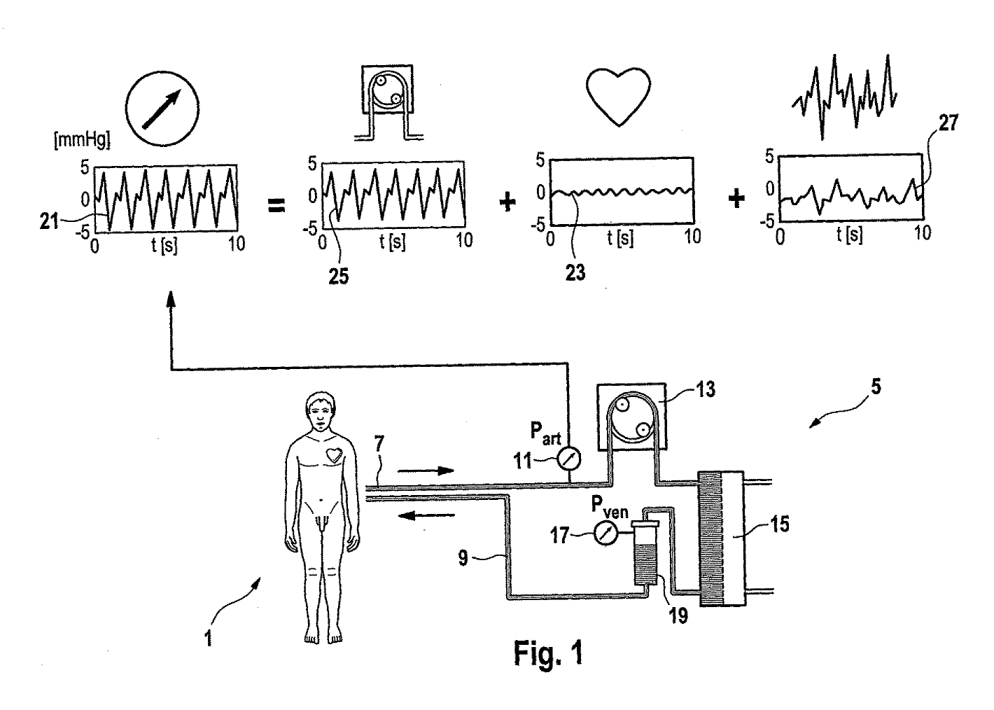

FIG. 1 shows in schematically simplified representation the principle of a heart signal extraction. A fistula (not shown) was applied to a patient 1 for the purpose of a blood treatment. The fistula is connected to a blood treatment apparatus 5 including an arterial branch 7 and a venous branch 9. The blood treatment apparatus 5 comprises a dialyzer 15, and on its arterial side a pressure sensor 11, a blood pump 13, on its venous side a pressure sensor 17 and a drip chamber 19. As is shown at the top of FIG. 1, the pressure signal 21 detected with the aid of the pressure sensor 11 includes the cardiac pressure signal 23 of the patient 1, a contribution 25 of the blood pump 13, and a measurement noise 27. According to the present invention, the amplitude of the cardiac pressure signal 23 may be determined based on the detected pressure signal 21. An evaluation of the amplitude of the determined cardiac pressure signal 23 is equally subject matter of the present invention, as was described in the foregoing.

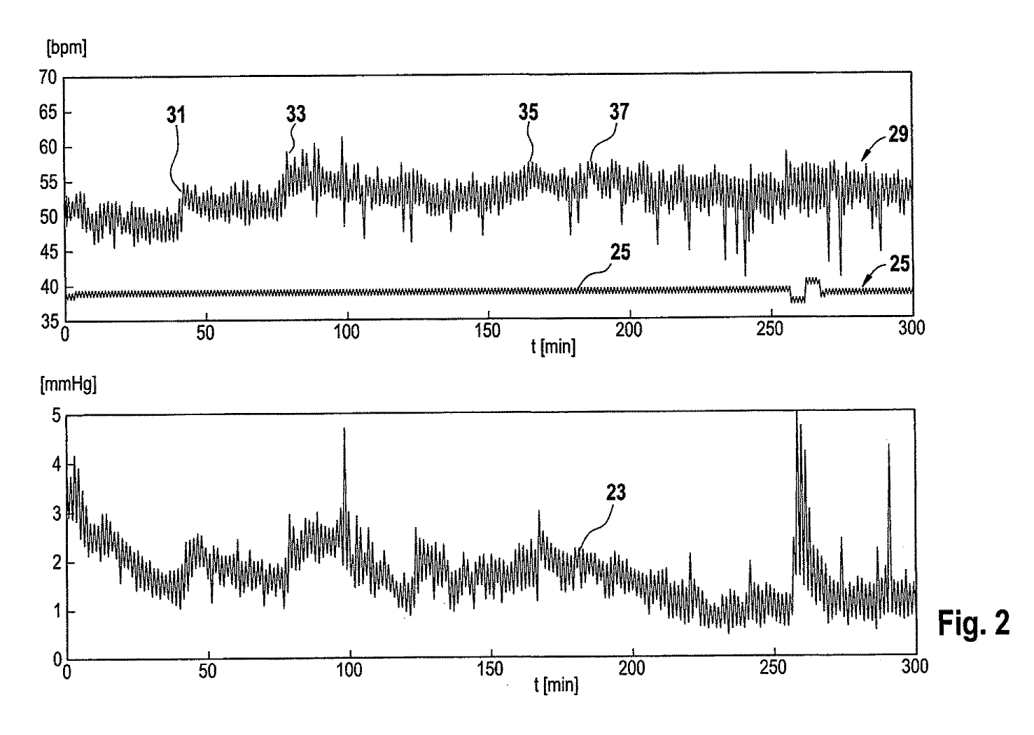

FIG. 2 shows graphs of a heart rate 29 (top, indicated in [bpm], i.e., beats per minute) and an amplitude of a cardiac pressure signal 23 (bottom) versus the duration of a blood treatment. The heart rate was validated by the inventors with the aid of a conventional EKG apparatus. In FIG. 2 the base frequency 25 of the blood pump of the used blood treatment apparatus having the designation 5008 by the enterprise Fresenius Medical Care is represented in addition. The evolution of the heart rate 29 exhibits several violent changes in FIG. 2. Thus, according to expectation, the heart rate 29 increases at the time 31 of the patient's awakening, at the time 33 of breakfast, at the time 35 of reaching the half-time of the treatment, or on the occasion of the physician's visit at the time 37. The cardiac pressure signal 23 is subject to equally clear trends. Its amplitude fluctuates between approximately 4 and 1 mmHg.

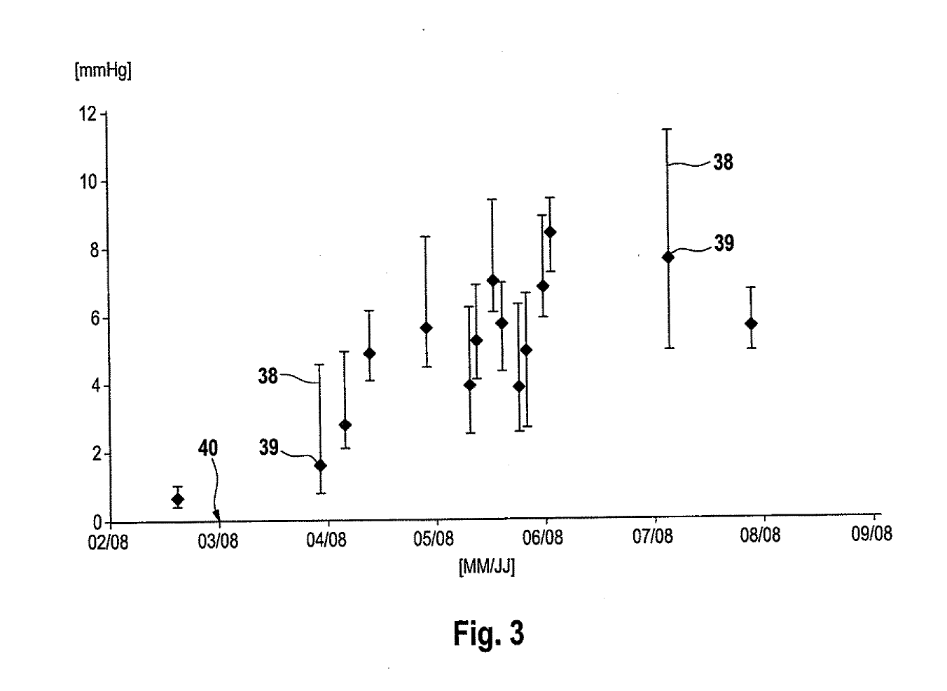

FIG. 3 shows values for the amplitudes of a cardiac pressure signal in mmHg over a time period between February (02/08) and September (09/08) of the year 2008. The bars 38 each represent the median 39 and the tenth and ninetieth percentile of the amplitudes of the cardiac pressure signal for a complete treatment. After the first measurement in February of 2008, a new placement of a fistula took place at a time 40 as a graft (previously a central venous catheter was used). The mean amplitude of the cardiac pressure signal subsequently continued to increase steadily over weeks, indicating a developing outflow stenosis of the fistula. Such outflow stenoses occur regularly with Goretex grafts. The time period observed in this case approximately represents the maturing period for the graft. It is possible to define a range in which the amplitude of the cardiac pressure signal should be situated in the long term. An outflow stenosis accompanied by an amplitude of the cardiac pressure signal of>20 mmHg may already clearly restrict the fistula flow. Such a range may be globally valid or may be determined anew for each patient. As is shown in FIG. 3, it is possible to correspondingly recognize variations at the vascular access with the aid of long-term monitoring of the amplitude of the cardiac pressure signal.

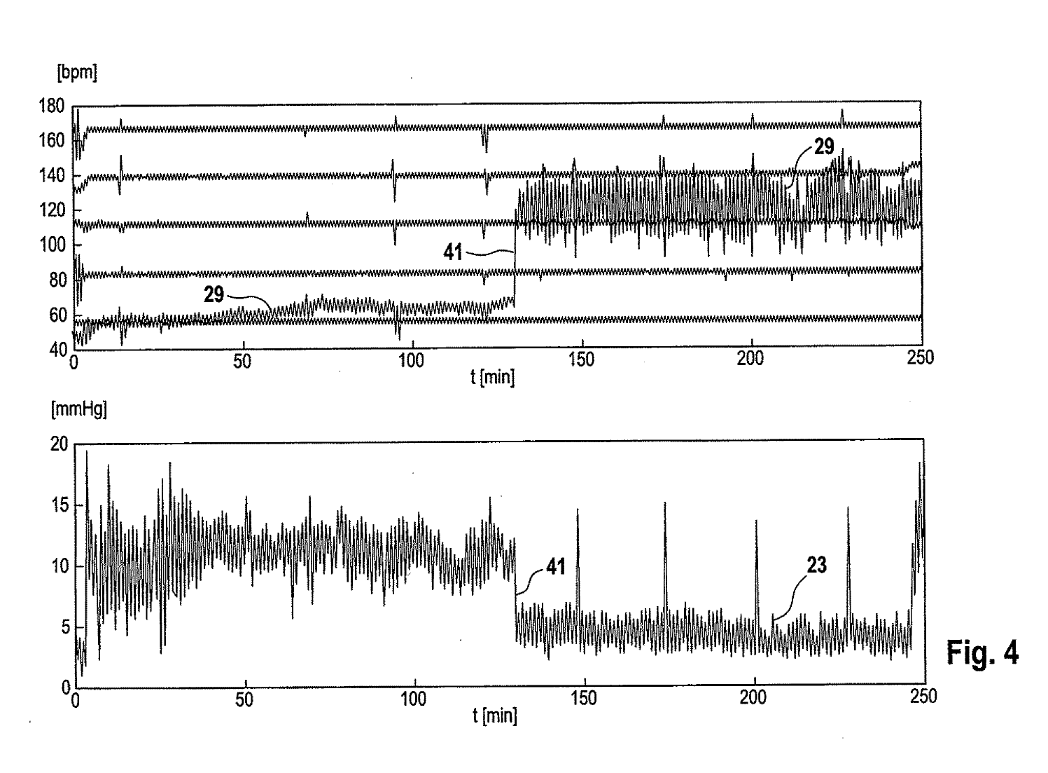

FIG. 4 shows graphs of a heart frequency 29 (top) and an amplitude of a cardiac pressure signal 23 (bottom) versus time. The violent change occurring in both graphs after approximately 130 minutes at the time 41 reflects a transition to intermittent atrial fibrillation. Such a process leads to a high, irregular pulse, as is visible at the top of FIG. 4,

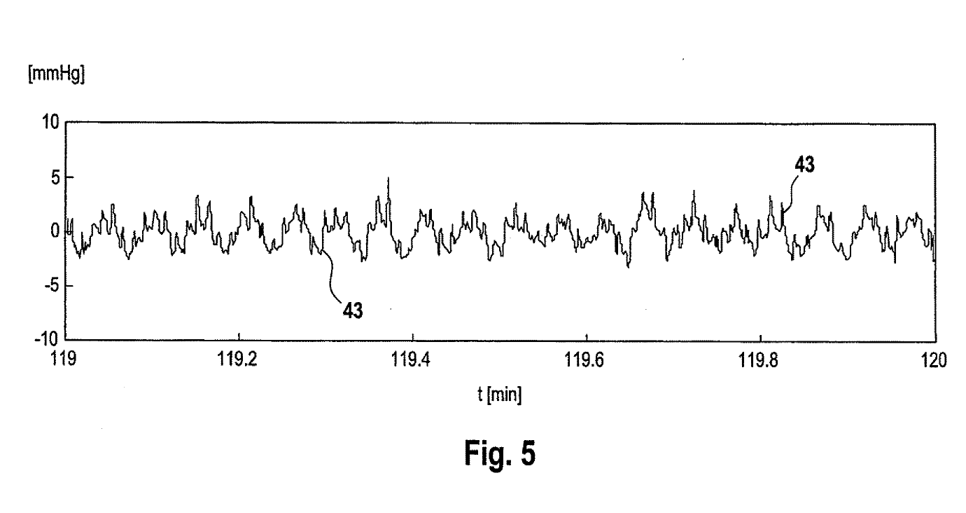

FIG. 5 is a graphical representation of a superposition 43 of influences of respiration and cardiac pressure signal versus time. The superposition 43 is composed of small peaks of the heartbeat and large fluctuations of the respiration. Particularly with catheterized patients, respiration may be represented by measurement in the right atrium. The intrathoracal pressure of the respiration may be detected in accordance with the representation of FIG. 5.

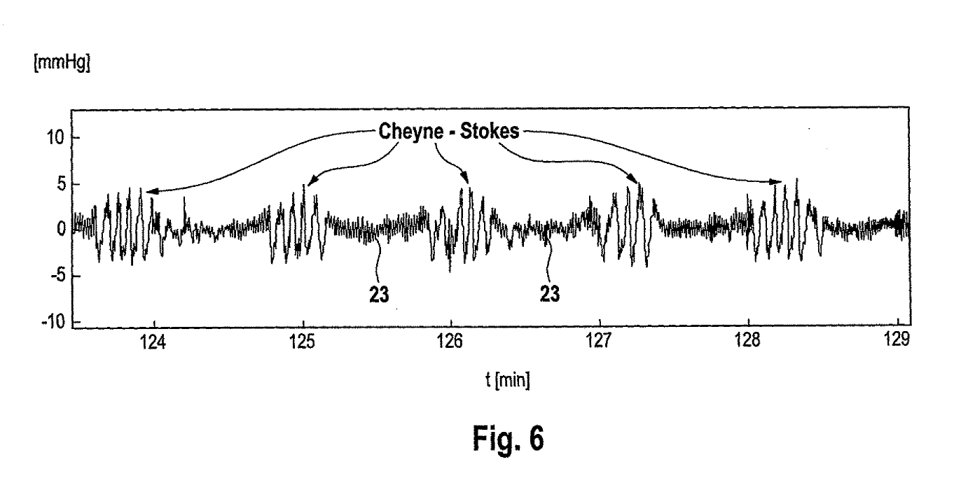

FIG. 6 shows a Cheyne-Stokes respiration, with five to six successive breaths taking place, followed by a respiration pause. In the respiration pause the heart pulsation is well discernible in the characteristic amplitude of the cardiac pressure signal 23.

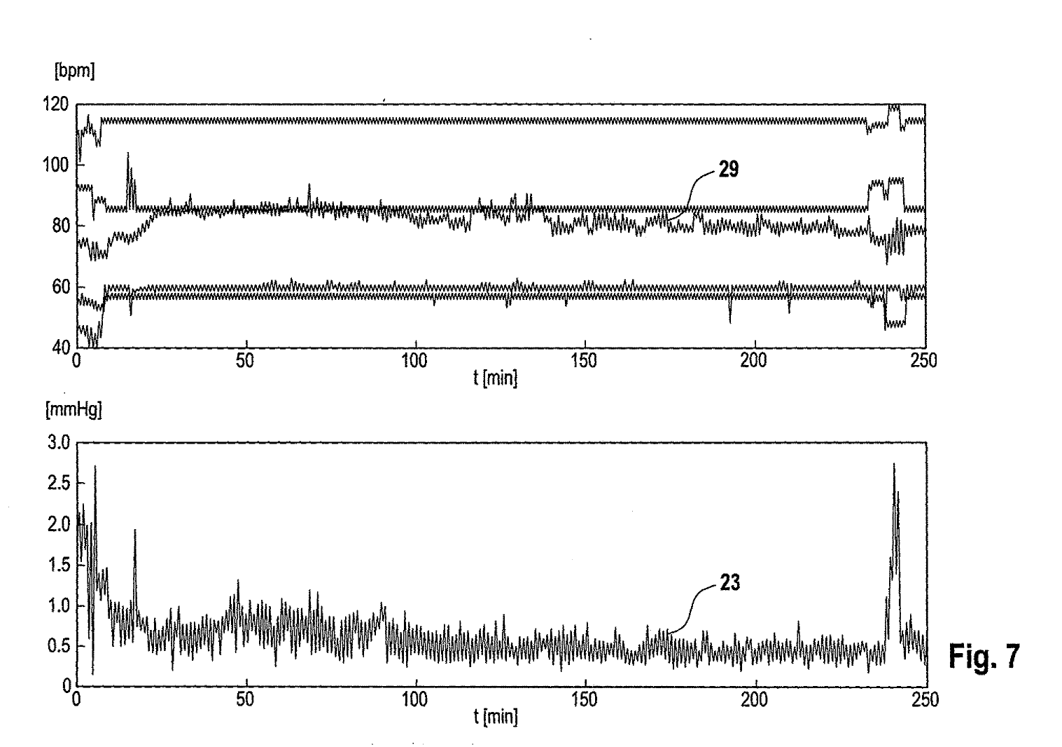

FIG. 7 is another graph showing a heart frequency 29 (top) and the amplitude of a cardiac pressure signal 23 (bottom) versus time. The corresponding data was obtained from a heart pacemaker patient. The heart pacemaker only “intervenes” occasionally, leading to the two different heart rates as represented.

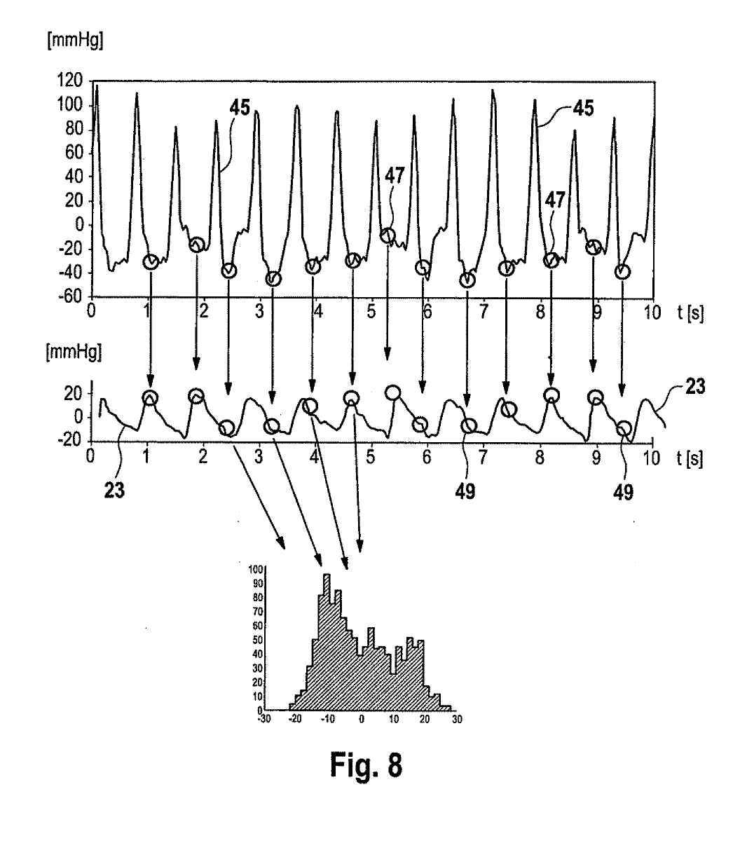

FIG. 8 schematically shows the extraction of a cardiac pressure signal 23 from a measurement signal 45 including not only the cardiac pressure signal but also a pump signal (top), and a corresponding histogram (bottom). The extraction of the cardiac pressure signal 23 takes place by detecting Hall sensor signals of a blood pump such as, e.g., a blood pump of a dialysis machine belonging to the machine generation bearing the designation 5008 by the company Fresenius Medical Care. The circles 47 indicate the Hall sensor-synchronous sampling. The circles 49 indicate the cardiac pressure signal at the time of the Hall sensor pulses. Sampling takes place at every Hall sensor pulse. The histogram represents the values of the circles 49. Standard deviations may here be taken as a measure for the intensity of the pulsation. The mean value in the histogram may furnish or enable a statement about the fistula pressure. For example, the average +/− of a standard deviation may serve as a measure for the cardiac pressure amplitude. What is also possible is the utilization of a percentile (10th, 90th, etc.), a percentile range, or combinations thereof.

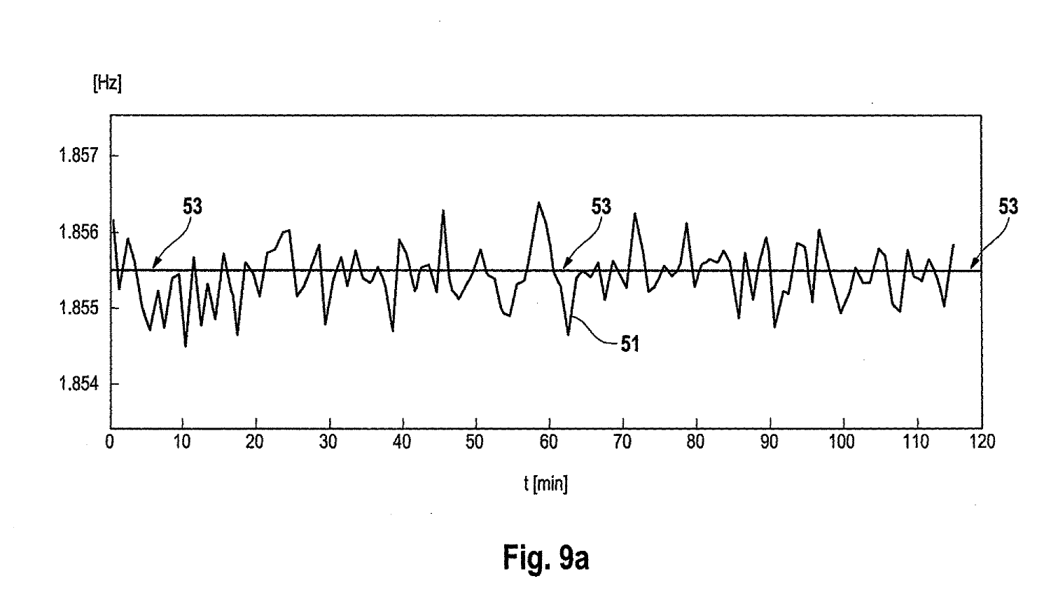

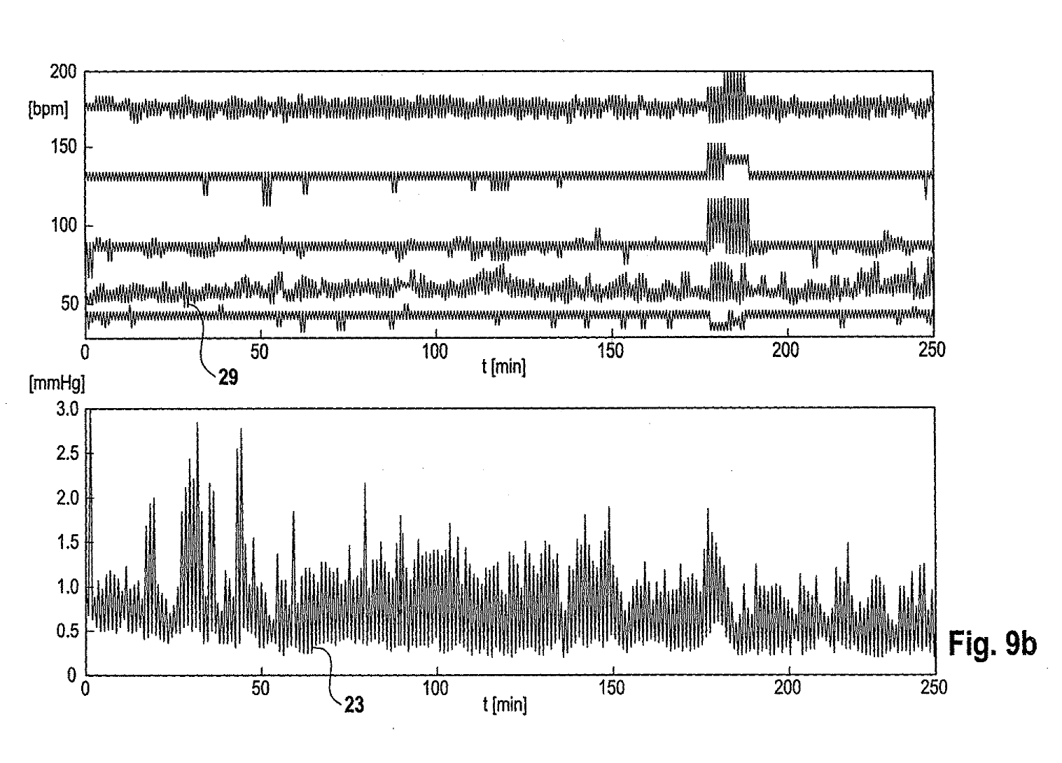

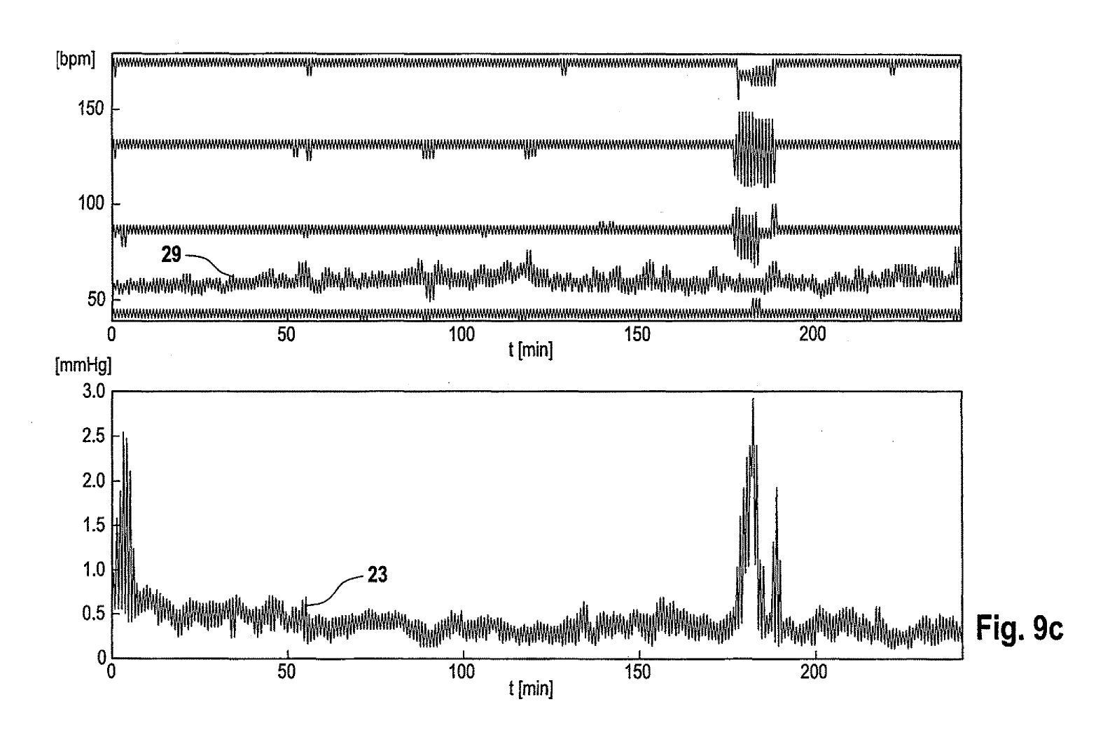

FIGS. 9a to 9c schematically show the difference between instances of time-synchronous (FIG. 9b) and angle-synchronous (FIG. 9c) sampling of the pump signal. In difference from the representation in FIG. 8, the arterial pressure signal may also be sampled angle-synchronously with a pump rotor. This may improve the extraction of the cardiac pressure signal, particularly if the rotation of the blood pump is not perfectly uniform, i.e., if the pump frequency 51 deviates from a target frequency 53, as is shown in FIG. 9a. The unit [Hz] represents the pump frequency.

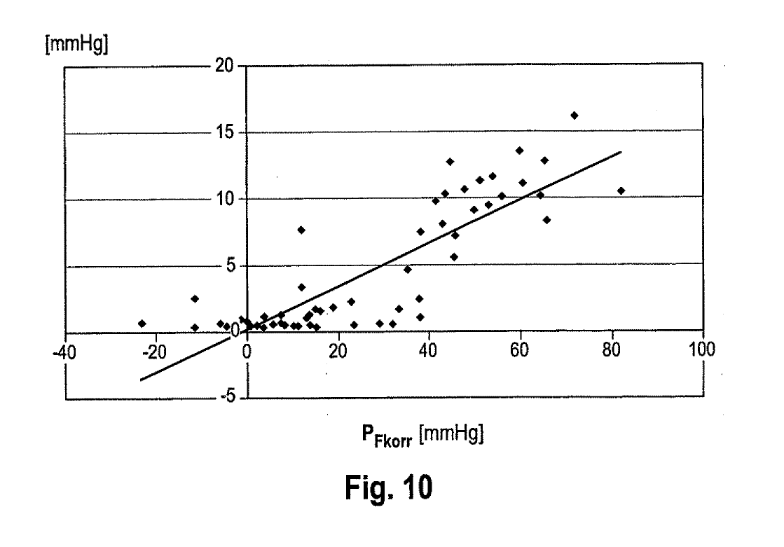

FIG. 10 shows a graph of acardiac pressure signal 23 versus a hydrostatically corrected fistula pressure PFkorr (at the arterial needle within the fistula) with values from more than 50 dialysis treatments. There is an interrelation between the fistula pressure and the amplitude of the cardiac pressure signal. This is on the one hand due to a reduction of the stroke volume: with a dropping supply of blood during a treatment the stroke volume equally becomes lower, leading to a raised heart rate by way of compensation. On the other hand this may be explained by a reduction of the fistula pressure. It is possible to infer a relative variation of the fistula pressure if the effect of the stroke volume is corrected via the heart rate, for example. The interrelation between fistula pressure and amplitude may also be founded in the elasticity of the fistula. A tightly filled fistula is not capable of further dilation and transmits heart pulses at less attenuation or nearly without attenuation. An empty, slack fistula attenuates the pulsation more strongly. By determining the amplitude at various speeds of the blood pump it is furthermore possible to infer the blood supply from the fistula and thereby perform an estimation of the fistula flow.

1-24. (canceled)

25. A method for determining and/or monitoring at least one physical condition of a patient, comprising:
evaluating an amplitude of a pressure signal so as to generate a statement concerning the condition.

26. The method according to claim 25, further comprising:
determining the amplitude of the pressure signal during a blood treatment of the patient with the aid of a blood treatment apparatus.

27. The method according to claim 26, wherein the blood treatment is a hemodialysis, a hemofiltration, or a hemodiafiltration.

28. The method according to claim 26, further comprising:
correcting the determined value of the amplitude of the pressure signal by a contribution of the blood treatment apparatus to the magnitude of the amplitude of the pressure signal.

29. The method according to claim 28, including the step of
correcting the determined value of the amplitude of the pressure signal as a function of a signal transmitted by a position sensor.

30. The method according to claim 29, wherein the position sensor is a Hall sensor.

31. The method according to claim 28, further comprising:
correcting the determined value of the amplitude of the pressure signal as a function of a rotary angle of a peristaltic blood pump of the blood treatment apparatus.

32. The method according to claim 25, further comprising:
evaluating the amplitude of the pressure signal by comparison to predetermined reference values.

33. The method according to claim 25, wherein the physical condition is a cardiovascular quantity.

34. The method according to claim 25, wherein the method is performed as an off-line method.

35. The method according to claim 25, wherein the physical condition relates to the patient's respiration.

36. The method according to claim 25, further comprising:
evaluating the amplitude of the pressure signal so as to observe a long-term trend of the physical condition.

37. The method according to claim 25, wherein the physical condition is a fistula transmissivity.

38. The method according to claim 25, further comprising:
evaluating the amplitude of the pressure signal as to tachycardia, bradycardia and/or hyper- or hypotensive episodes.

39. The method according to claim 25, further comprising:
establishing a classification of a patient.

40. The method according to claim 25, wherein the physical condition is a heart rate.

41. The method according to claim 25, wherein the physical condition is an arrhythmia.

42. The method according to claim 25, wherein the physical condition is the operation of a heart pacemaker.

43. The method according to claim 25, where in the evaluating is performed by a CPU.

44. An apparatus for determining and/or monitoring a physical condition of a patient, comprising:
at least one data processor configured to evaluate the amplitude of a pressure signal.

45. The apparatus according to claim 44, wherein the physical condition of the patient is at least one cardiovascular quantity of the patient.

46. The apparatus according to claim 44, further comprising:
at least one means for determining an amplitude of the cardiac pressure signal.

47. The apparatus according to claim 44, further comprising:
at least one means for correcting the value of the amplitude of the pressure amplitude signal by a contribution of a blood treatment apparatus to the magnitude of the amplitude of the pressure signal as a function of a signal of at least one position sensor and/or as a function of the rotary angle of a blood pump of the blood treatment apparatus.

48. The apparatus according to claim 47, wherein the at least one position sensor includes at least one Hall sensor.

49. The apparatus according to claim 44, further comprising:
means for supplying reference values.

50. The apparatus according to claim 44, further comprising:
output means for outputting a result of the evaluation.

51. A treatment apparatus, comprising:
at least one apparatus according to claim 44.

52. The treatment apparatus according to claim 51, wherein the treatment apparatus has the form of a blood treatment apparatus.

53. the treatment apparatus according to claim 52, where the blood treatment apparatus is a hemofiltration apparatus, hemodialysis apparatus, or hemodiafiltration apparatus.
